(19) United States
(12) Patent Application Publication
(10) Pub. No.: US 2026/0360350 A1
(43) Pub. Date: Nov. 12, 2026

(54) SYSTEM AND METHOD FOR TRANSLATING MULTIMODAL BIOMETRIC SIGNALS INTO ADAPTIVE EXPRESSIVE OUTPUT

(71) Applicant: Kristy Violette, Sarasota, FL (US)

(72) Inventor: Kristy Violette, Sarasota, FL (US)

(21) Appl. No.: 19/668,175

(22) Filed: May 5, 2026

Related U.S. Application Data
(60) Provisional application No. 63/803,768, filed on May 11, 2025.

(51) Int. Cl. G10L 13/033; A61B 5/00; A61B 5/16; G06F 3/01

(52) U.S. Cl. CPC G10L 13/033; A61B 5/165; A61B 5/681; A61B 5/7221; A61B 5/741; A61B 5/7455; G06F 3/015; G06F 3/017

(57) ABSTRACT

An arm-mounted communication system fuses autonomic, neuromuscular, emotional, and kinematic signals—photoplethysmography, galvanic skin response, electromyography, and inertial data—on an edge processor that executes a user-specific baseline model. Optionally, emerging sensors such as graphene impedance tattoos, microfluidic sweat cortisol electrodes, and non-invasive neural sensors contribute additional features. Incoming vectors are mapped to intent and emotional states; the processor selects a personalized expressive output comprising synthesized speech, vibrotactile feedback, and/or gesture cues. A remote Vocal Coach platform operated by certified specialists receives anonymized performance metrics and returns tuning parameters. Raw biometrics remain locally encrypted and are auto-deleted after thirty days. Tiered support plans incorporate a scholarship-management module that unlocks premium features upon third-party funding verification. The hypoallergenic, sub-30-gram device defaults to short check-ins; continuous sensing is disabled unless enabled. Federated learning, age-progressive voice updates, and multimodal emerging sensors provide lifelong adaptability.

### CROSS-REFERENCE TO RELATED APPLICATIONS

This application claims the benefit under 35 U.S.C. § 119(e) of U.S. Provisional Patent Application No. 63/803,768, filed May 11, 2025, the entire disclosure of which is incorporated herein by reference.

### 1. FIELD

The disclosure relates to assistive communication technology and, more particularly, to systems and methods that translate multimodal biometric signals into adaptive expressive output for pre-verbal, nonverbal, or speech-delayed individuals.

### 2. BACKGROUND

Augmentative-and-alternative communication devices often rely on rigid symbol grids or camera-based eye tracking, demanding fine motor control and substantial caregiver programming. Existing stress-monitoring wearables typically transmit sensitive biometric data to cloud servers, raising privacy concerns and limiting adoption among privacy-conscious users.

### 3. SUMMARY

Disclosed is a hypoallergenic, sub-30-gram arm-mounted communication system that fuses autonomic (PPG/HRV), emotional (GSR), neuromuscular (EMG), kinematic (IMU), and optional emerging sensors-graphene impedance tattoos, microfluidic sweat cortisol electrodes, and non-invasive neural sensors-into a single feature vector processed by an edge-resident personalized baseline model. The system outputs synthesized speech, vibrotactile feedback, or gesture cues according to the classified intent/emotional state. A remote Vocal-Coach platform receives anonymized performance metrics and returns tuning parameters, while a scholarship-management module unlocks premium support tiers for qualified users. All raw biometrics remain on-device, encrypted, and are deleted after thirty days. Continuous sensing is disabled by default; federated-learning routines and age-progressive voice updates ensure lifelong adaptability. The system integrates with related biometric platforms, such as those disclosed in U.S. patent application Ser. No. 19/194,650 (filed Apr. 30, 2025), U.S. Provisional Patent Application No. 63/788,032 (filed Apr. 13, 2025), and U.S. Provisional Patent Application No. 63/802,824 (filed May 9, 2025), enhancing performance and recovery applications.

### 4. BRIEF DESCRIPTION OF THE DRAWINGS

FIG. 1—Exploded Perspective View of the Ace Unlock Arm-Mounted Wearable System

Illustrating internal sensor placement zones. Labeled components include: photoplethysmography (PPG) sensor, galvanic skin response (GSR) electrodes, electromyography (EMG) array, inertial measurement unit (IMU), optional graphene impedance patch (180), microfluidic sweat-sensing array (195), and ultrasound array (190, dashed outline indicates optional module).

FIG. 2—Signal-Processing Pipeline from Raw Biometric Sensor Input to Expressive Output

Sensor streams are fused in a feature extraction layer, classified by an on-device neural network against a personalized baseline model, and routed to speech, haptic, or gesture output modules. A dotted input arrow represents optional emerging sensor modalities.

FIG. 3—Feedback and Refinement Loop Between the User Device, Caregiver, and Vocal Coach Platform

Caregivers tag session outputs, which are transmitted as anonymized metrics. Certified specialists update tuning parameters via encrypted packets. A scholarship API gateway unlocks premium support features based on verified third-party funding.

FIG. 4—Voice-Library User Interface Showing Age, Gender, and Dialect Selectors Plus Confidence Gauge for Personalized Expressive Outputs

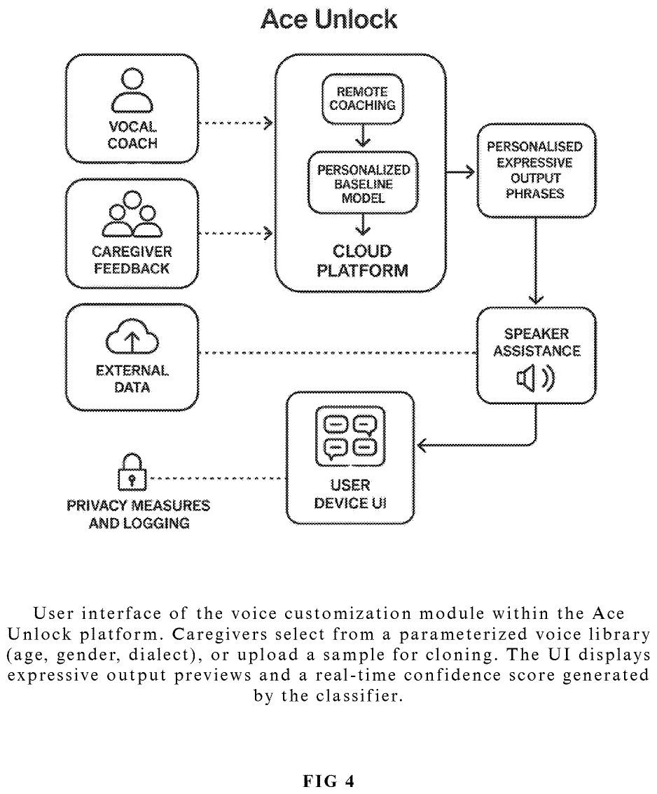

FIG. 5—Privacy and Data-Retention Workflow

Biometric streams are encrypted locally and stored in a secure device memory. Daily summaries are anonymized and transmitted for remote analysis. Raw data are auto-deleted after 30 days unless retention is explicitly authorized. Federated learning updates are derived from encrypted gradient deltas only.

### 5. DETAILED DESCRIPTION

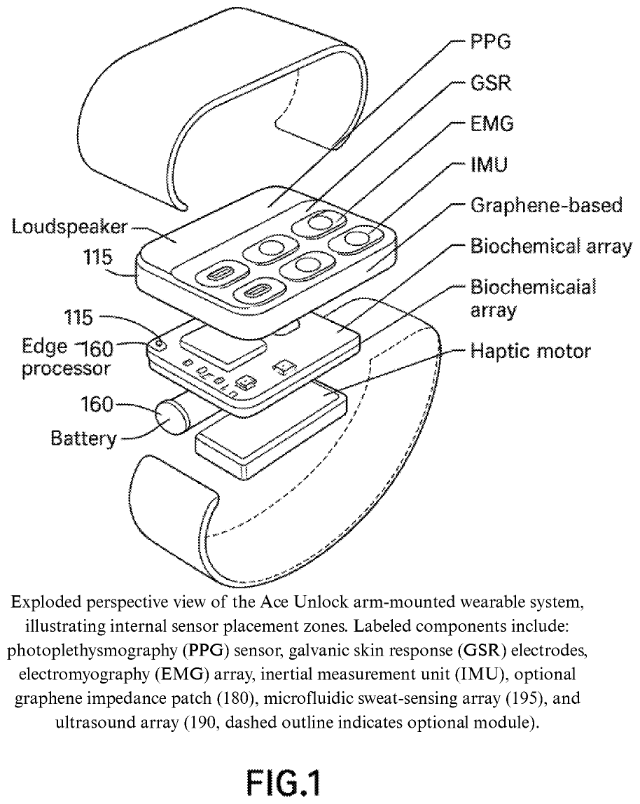

### 5.1 Hardware Architecture (FIG. 1)

The wearable comprises a polycarbonate core over-molded with hypoallergenic silicone, maintaining a mass <30 g. Core sensors: PPG (256 Hz), GSR (32 Hz), eight-channel dry-electrode EMG (1 kHz), and a 9-DOF IMU (200 Hz). All sensor components, including optional graphene tattoos and biochemical arrays, are engineered to minimise tactile, thermal, and visual stimuli, complying with paediatric occupational-therapy guidelines for sensory-sensitive users.

### Optional Emerging Sensors

Graphene electronic-tattoo 180 (<5 μm) sampling skin impedance at 100 Hz for sympathetic-arousal indices.
Microfluidic sweat array 195 delivering≈1 μL min⁻¹ to an amperometric cortisol electrode sampled every 10 min.
Hybrid “StressFit” electrodes 130a combining EMG pads with integrated cortisol/lactate wells.
Ultrasound array 190 (optional auxiliary module, dashed in FIG. 1) 16-element piezo (<10 g) imaging muscle fascicles at 10 MHz, streaming median echo intensity every 5 s. Ultrasound arrays are optional auxiliary modules, potentially implemented as forearm patches to minimize arm-mounted bulk, ensuring sensory compatibility.
Neural-activity sensor 170: eight dry EEG electrodes (0.5-40 Hz, 256 Hz sampling) or a two-channel fNIRS strip (760 nm/850 nm).

Auxiliary embodiments may include a 77 GHz mmWave radar node and photonic-yarn garments; derived metrics stream via BLE 5.3. Photonic textiles are optional auxiliary inputs, implemented in garments to prioritize sensory comfort for neurodivergent users, with derived pulse and stretch metrics streamed via BLE.

### 5.2 Personalised Baseline Model

Three 90 s enrolment sessions seed a CNN-LSTM baseline; optional neural alpha-asymmetry and ultrasound muscle-tone indices are concatenated where present. Model retrains locally after each check-in; nightly federated-learning aggregates encrypted gradients.

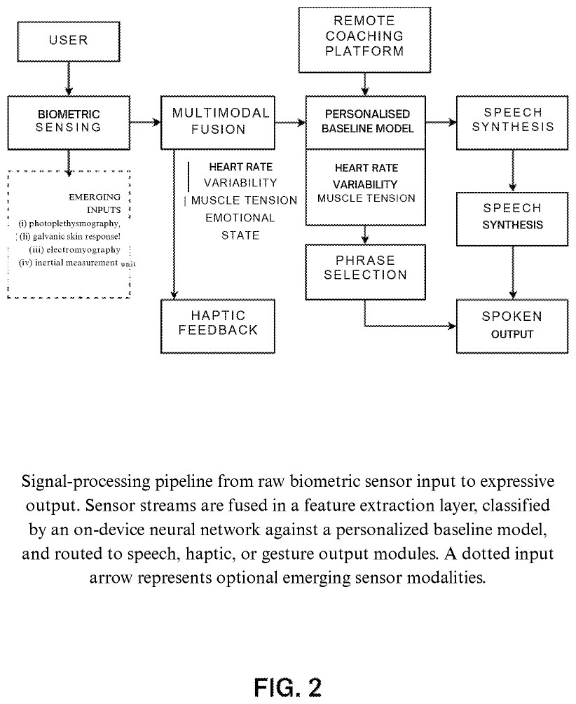

### 5.3 Intent Classification & Confidence Scoring (FIG. 2)

Feature Fusion Layer 200 concatenates autonomic, neuromuscular, biochemical, neural, and kinematic inputs (zero-padded if absent). The model outputs probabilities for comfort, request-assist, frustration, excitement, and a confidence score; scores <0.60 trigger fallback haptic prompts. Intent classification adheres to ethical guidelines developed with pediatric ethicists, ensuring outputs reflect user intent without misrepresentation.

### 5.4 Expressive Output Generation

Tacotron-style TTS produces cloned or library voices with age-progression. LR-A motor delivers patterned haptics; gesture cues display on a paired smart device. End-to-end latency ≤150 ms.

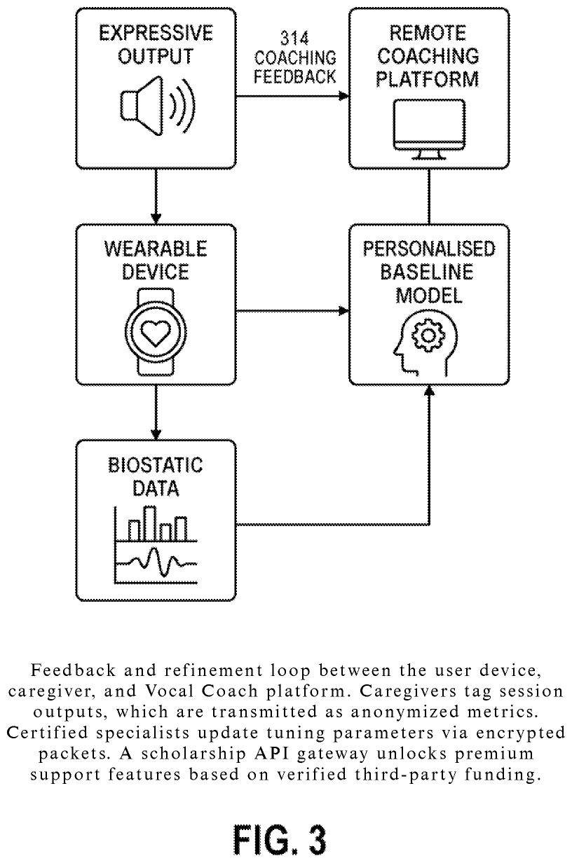

### 5.5 Remote Vocal-Coach Platform (FIG. 3)

Certified specialists review anonymised dashboards and, using caregiver feedback, calibrate baseline models to maintain ≥95% expressive-output accuracy.

### 5.6 Tiered Support & Scholarship Integration

Ace Essentials, Ace Concierge, and Ace Clinical tiers; scholarship module verifies funding via OAuth 2.0 to unlock Concierge features. Endpoint definitions appear in Appendix A, Table 1.

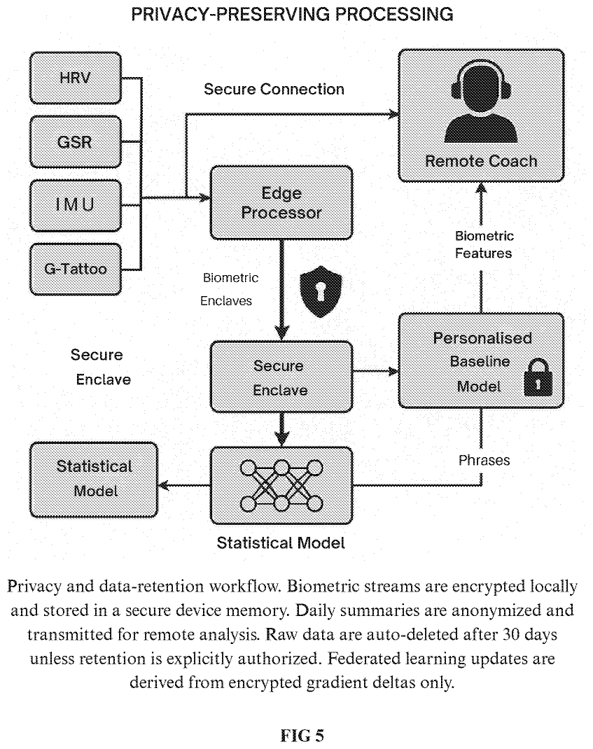

### 5.7 Privacy & Security (FIG. 5)

AES-256 encryption on-device; 30-day auto-delete; BLE pairing via ECDH; framework vetted by paediatric-ethics panel.

### 5.8 Optional Continuous Mode

Enabled only when caregiver toggles on, battery ≥20%, and runtime <12 h.

### 5.9 Example Use Cases

1. Preschooler communicates “break” via cloned voice; high-confidence alert.
2. TBI adult orders lunch using vibrotactile confirm+dialect voice.
3. Scholarship classroom deploys ten devices; coach tunes dialect voices.

### 6. INDUSTRIAL APPLICABILITY

Enables emotionally attuned, privacy-preserving communication across home, clinical, educational, and rehabilitative settings.

### 7. REFERENCE NUMERAL LIST

130a—Hybrid “StressFit” electrodes combining EMG pads with integrated cortisol/lactate wells, as part of the electromyography sensor suite.
170—Neural-activity sensor, comprising eight dry EEG electrodes or a two-channel fNIRS strip, for optional cognitive intent detection.
180—Graphene electronic-tattoo impedance sensor, sampling skin impedance at 100 Hz for sympathetic-arousal indices.
190—Ultrasound array, an optional auxiliary module with 16 piezo elements for imaging muscle fascicles at 10 MHz, depicted as a dashed outline in FIG. 1.
195—Microfluidic sweat-sensing array, delivering 1 μL min⁻¹ to an amperometric cortisol electrode for stress biomarker analysis.
200—Feature Fusion Layer, an on-device processing module that concatenates multimodal biometric inputs for intent classification, as shown in FIG. 2.

### 8. APPENDIX A—SCHOLARSHIP API ENDPOINT TABLE

| Endpoint | Method | Parameters | Description |
|---|---|---|---|
| /auth/ | POST | client_id, | Requests an OAuth |
| | | client_secret, | 2.0 access token using |
| | | grant_type, code | third-party funding |
| | | | credentials. |
| /funding/verify | GET | token, | Verifies funding |
| | | funding_id | eligibility with third- |
| | | | party provider (e.g., |
| | | | Step Up for Students) |
| | | | using the access |
| | | | token. Returns |
| | | | eligibility status (e.g., |
| | | | {“eligible”: true}). |
| /features/unlock | POST | access_token, | Unlocks premium |
| | | user_id, | features (e.g., Ace |
| | | feature_id | Concierge) for the |
| | | | specified user upon |
| | | | successful funding |
| | | | verification. |
| /funding/status | GET | access_token, | Retrieves the funding |
| | | user_id | status and unlocked |
| | | | features for a user, |
| | | | including expiration |
| | | | dates. |
| /auth/refresh | POST | refresh_token, | Refreshes the OAuth |
| | | client_id | 2.0 access token to |
| | | | maintain secure API |
| | | | access. |

Notes:
All endpoints use HTTPS for secure communication, with AES-256 encryption for data in transit, consistent with the privacy framework (Section 5.7).
The API supports extensibility for additional funding providers (e.g., Medicaid, United Way) via configurable funding_id parameters.
Error handling includes standard HTTP status codes (e.g., 401 Unauthorized for invalid tokens, 200 OK for successful requests).

What is claimed is:

1. A wearable system for generating adaptive expressive output, comprising:
(a) a housing configured to be worn on the body of a user;
(b) a sensor suite disposed within or on the housing, the sensor suite configured to acquire at least two concurrent signal streams from different physiological, biometric, neuromuscular, behavioral, kinematic, or other measurable modalities of the user;
(c) a non-transitory memory storing instructions and a personalized baseline model derived from prior signals of the user;
(d) an edge processor coupled to the sensor suite and to the memory, the edge processor configured to:
(i) receive the at least two concurrent signal streams from the sensor suite;
(ii) compare the received signal streams against the personalized baseline model to classify a state of the user, the state representing at least one of an emotional state, an intent state, and a communication state;
(iii) select, based on the classification, an adaptive expressive output from a set comprising synthesized speech, a vibrotactile haptic pattern, and a gesture-command cue; and
(iv) cause the selected adaptive expressive output to be presented through at least one output module integrated with or coupled to the housing;
wherein raw biometric signal values acquired by the sensor suite are not transmitted in raw form to any system external to the housing.

2. The system of claim 1, wherein the sensor suite comprises at least two of:
(a) a photoplethysmography sensor configured to derive heart-rate variability;
(b) a galvanic-skin-response sensor configured to measure electrodermal activity;
(c) an electromyography sensor configured to detect neuromuscular signals; and
(d) an inertial-measurement unit configured to detect motion or gesture.

3. The system of claim 1, wherein the sensor suite further comprises at least one sensor selected from the group consisting of:
(a) a graphene-based skin-impedance sensor;
(b) a microfluidic electrochemical sweat-analysis sensor;
(c) an electroencephalography or near-infrared spectroscopy neural-activity sensor; and
(d) an ultrasound array configured to image muscle fascicles.

4. The system of claim 1, wherein the memory stores raw biometric signal values only in encrypted form, and wherein the edge processor is configured to automatically delete the raw biometric signal values after a predefined retention period.

5. The system of claim 1, wherein the edge processor is further configured to:
(a) compute a confidence score associated with the classification of the state of the user; and
(b) when the confidence score is below a predefined threshold, cause a predetermined fallback expressive output to be presented in lieu of, or in addition to, the selected adaptive expressive output.

6. The system of claim 1, wherein the edge processor disables continuous sensing by default and enables continuous sensing only upon explicit authorization received through an authorized credential.

7. The system of claim 1, wherein the synthesized speech is generated by at least one of:
(a) cloning a voice sample provided by a caregiver or other authorized party; and
(b) selecting a voice from a parameterized library defining attributes including age, gender, and dialect.

8. The system of claim 7, wherein the synthesized speech is automatically updated at predefined developmental milestones to reflect changing vocal characteristics of the user.

9. The system of claim 1, wherein the at least one output module comprises a haptic actuator configured to deliver distinct vibration motifs mapped to at least four discrete intent categories.

10. The system of claim 1, wherein the sensor suite comprises an inertial-measurement unit configured to differentiate between hand gestures indicative of “yes,” “no,” and “help.”

11. The system of claim 1, wherein the housing is configured to be worn on a limb of the user, is formed at least in part of hypoallergenic material, and collectively weighs no more than thirty grams.

12. A method of generating adaptive expressive output for a user, comprising:
(a) acquiring, by a sensor suite of a wearable device worn on the body of the user, at least two concurrent signal streams from different physiological, biometric, neuromuscular, behavioral, kinematic, or other measurable modalities of the user;
(b) comparing, on an edge processor of the wearable device, the at least two concurrent signal streams against a personalized baseline model stored on the wearable device, to classify a state of the user representing at least one of an emotional state, an intent state, and a communication state;
(c) selecting, based on the classification, an adaptive expressive output from a set comprising synthesized speech, a vibrotactile haptic pattern, and a gesture-command cue;
(d) causing the selected adaptive expressive output to be presented; and
(e) maintaining raw biometric signal values acquired by the sensor suite within the wearable device, without transmitting the raw biometric signal values in raw form to any system external to the wearable device.

13. The method of claim 12, further comprising storing raw biometric signal values acquired by the sensor suite only in encrypted form on the wearable device, and automatically deleting the raw biometric signal values after a predefined retention period.

14. The method of claim 12, further comprising:
(a) computing a confidence score associated with the classification; and
(b) when the confidence score is below a predefined threshold, causing a predetermined fallback expressive output to be presented in lieu of, or in addition to, the selected adaptive expressive output.

15. The method of claim 12, further comprising updating the personalized baseline model on the wearable device based on a plurality of subsequent check-in sessions performed by the user, without transmitting raw biometric signal values acquired during the check-in sessions to any system external to the wearable device.

16. A wearable system for generating adaptive expressive output with privacy-preserving on-device processing, comprising:
(a) a housing configured to be worn on the body of a user;
(b) a sensor suite disposed within or on the housing, the sensor suite configured to acquire signals from one or more physiological, biometric, neuromuscular, behavioral, kinematic, or other measurable modalities of the user;
(c) a non-transitory memory storing a personalized baseline model derived from prior signals of the user;
(d) an edge processor coupled to the sensor suite and to the memory, the edge processor configured to:
(i) update the personalized baseline model on the wearable system based on a plurality of check-in sessions performed by the user, without transmitting raw biometric signal values acquired during the check-in sessions to any system external to the housing;
(ii) classify a state of the user, the state representing at least one of an emotional state, an intent state, and a communication state, by comparing current signals from the sensor suite against the updated personalized baseline model; and
(iii) cause an adaptive expressive output to be generated based on the classification, the adaptive expressive output comprising at least one of synthesized speech, a vibrotactile haptic pattern, and a gesture-command cue; and
(e) a communications interface configured to transmit, to a remote system, at least one of (i) anonymized metrics derived from the signals acquired by the sensor suite and (ii) gradient-only updates of the personalized baseline model, while excluding raw biometric signal values from the transmission.

17. The system of claim 16, wherein the gradient-only updates of the personalized baseline model are aggregated across a plurality of devices via a federated-learning routine, such that raw biometric signal values acquired by the sensor suite of any device are not shared with the federated-learning routine.

18. The system of claim 16, wherein the personalized baseline model is updated after at least three check-in sessions of the user.

19. The system of claim 16, further comprising a remote coaching platform configured to receive the anonymized metrics, generate updated model parameters based on the anonymized metrics, and transmit the updated model parameters to the wearable system, wherein no raw biometric signal values are transmitted from the wearable system to the remote coaching platform.

20. The system of claim 16, wherein the signals acquired by the sensor suite include at least two of: heart-rate variability, electrodermal activity, neuromuscular activity, motion or gesture, skin impedance, biochemical sweat composition, and neural activity.
